(19) United States
(12) Patent Application Publication
(10) Pub. No.: US 2020/0316091 A1
(43) Pub. Date: Oct. 8, 2020

(54) METHOD FOR PROVIDING CONTRACEPTION

(71) Applicant: LABORATOIRE HRA-PHARMA, Châtillon (FR)

(72) Inventors: Camille BANH, Les Lilas (FR); Pascale BORENSZTEIN, Paris (FR); Annabelle GLASIER, Edinburgh (GB); Delphine LEVY-GOMPEL, Shanghai (CN); Catherine MONTEIL, Paris (FR)

(21) Appl. No.: 16/839,353

(22) Filed: Apr. 3, 2020

(30) Foreign Application Priority Data
Apr. 5, 2019 (EP) 19305452.5

(51) Int. Cl. A61K 31/57; A61K 31/566; A61K 9/00; A61K 9/70; A61P 15/18

(52) U.S. Cl. CPC A61K 31/57; A61K 31/566; A61P 15/18; A61K 9/7023; A61K 9/0036

(57) ABSTRACT

The invention relates to a method for providing contraception in a woman, wherein the woman is subjected to hormonal combined contraceptive but experiments at least one misuse of said hormonal combined contraceptive and wherein the method comprises administering ulipristal acetate or a metabolite thereof within 120 hours after said at least one misuse and resuming the hormonal combined contraceptive within 24 hours after the intake of ulipristal acetate in said woman.

### FIELD OF THE INVENTION

The present invention relates to a method for providing contraception in a woman who is exposed to a suspected failure of her regular combined hormonal contraception.

### TECHNOLOGICAL BACKGROUND

Combined hormonal contraception refers to contraceptive methods based on the combination of an estrogen and a progestogen. The most popular combined contraceptives are Combined Oral Contraceptive Pills also referred to COCs or COCPs. These pills are designed to be taken every day. Most dosage regimen are based on a 28-day cycle comprising a first period of 21 days wherein the woman takes a daily pill containing both an estrogen and a progestogen, followed by a 7-day free-hormone period wherein the woman takes no pill or takes a daily placebo. COCs are currently used by more than 100 million women worldwide and by almost 12 million women in the United States. COCs, and more generally combined hormonal contraceptives, are highly effective to prevent pregnancy when perfectly used. However, lack of compliance with the posology specified in the labelling exposes the women to potential unintended pregnancy. For instance, ovulation may occur in women who miss one or several consecutive daily pills, in particular within the first week of the 28-day cycle. In that case, the clinical recommendation is generally to take an emergency contraceptive as soon as possible after the missed pill, especially when the missed pill occurs during the first week of the cycle. As of today, ulipristal acetate and levonorgestrel are the two main drugs authorized in the United States and in Europe for providing emergency contraception. However, the current label of Ella® (ulipristal acetate) in the US states that ulipristal acetate and progestin-containing contraceptives may interact and decrease the effectiveness of both products. The FDA further advises women to wait at least 5 days after taking ulipristal acetate to resume or start oral contraceptives. Similarly, the World Health Organization (WHO) advises that following use of emergency contraception with ulipristal acetate, women may “resume or start any progestogen containing method (either combined hormonal contraception or progestogen only contraceptives) on the 6th day after taking UPA” (WHO, Emergency Contraception, Fact sheets, February 2018). The Faculty of Sexual and Reproductive Healthcare Clinical Effectiveness Unit (FSRH CEU) even recommends using levonorgestrel rather than ulipristal acetate following a suspected failure of hormonal combined contraception.

These recommendations are in line with the findings of Edelman et al. (Contraception, 2018, 98, 463-466) who conducted a clinical trial to assess the impact of the early initiation of COC after ulipristal acetate intake. Edelman et al. showed that starting COC two days after ulipristal acetate intake significantly impairs efficacy of ulipristal acetate to inhibit or delay ovulation. In other words, women who resumed the COC two days after ulipristal acetate intake were at a higher risk of unintended pregnancy than women who did not.

### SUMMARY OF THE INVENTION

The invention relates to a method for providing contraception in a woman, wherein:
the woman is under hormonal combined contraceptive which is a combined oral contraceptive pill (COC) but experiments a suspected failure of contraception due to a misuse of said COC,
the misuse is at least one missed daily intake of the COC and
wherein the method comprises:
   administering ulipristal acetate or a metabolite thereof in said woman, said administration being performed after the misuse of the COC and within 120 h after an unprotected intercourse occurring in the same menstrual cycle as the misuse and
   resuming the hormonal combined contraceptive within about 24 hours after the intake of ulipristal acetate or a metabolite thereof in said woman.

In some embodiments, the woman has an intercourse within about 120 h before or after the misuse of the COC in said woman. Ulipristal acetate or a metabolite thereof is preferably administered at a dosage of 30 mg, orally, to the woman. In some embodiments, one single administration of ulipristal acetate or a metabolite thereof is performed to the woman.

The COC may comprise levonorgestrel as progestogen and ethinyl estradiol as estrogen.

In some embodiments, the method of contraception is characterized in that:
the misuse is missing at most three daily consecutive intakes of the COC,
ulipristal acetate or a metabolite thereof is administered within about 24 h after the last missed intake of the COC, and
the COC is resumed within about 18 h, preferably within about 12 h, after the intake of ulipristal acetate or a metabolite thereof.

Preferably, the woman has been treated with the COC for at least one menstrual cycle before the misuse of said COC.

In some embodiments, the COC is resumed the same day as the intake of ulipristal acetate or a metabolite thereof and/or within about 12 hours after the intake of ulipristal acetate or a metabolite thereof.

In another embodiment, ulipristal acetate or a metabolite thereof is administered within about 72 hours after the misuse of the COC.

The metabolite of ulipristal acetate may be selected from the group consisting of CDB-3877, CDB-3963, CDB-3236 and CDB-4183.

The method of the invention may be further for decreasing the risk of, or inhibiting, ovulation in the woman following the misuse of the COC in the remaining part of her menstrual cycle.

Another object of the invention is a method for providing contraception in a woman comprising orally administering an amount of 30 mg of ulipristal acetate within 120 hours after at least one misuse of a combined hormonal contraceptive that is a combined oral contraceptive (COC) and resuming the intake of the COC within 24 hours after the intake of ulipristal acetate in said woman. Typically, the woman has been treated with the COC for at least one menstrual cycle and wherein the misuse of the COC is at least one missed intake of said COC. In some embodiments, the woman misses at most three consecutive daily intakes of the COC. The COC may comprise ethinyl estradiol as estrogen and levonorgestrel as progestogen. In some embodiments, ulipristal acetate is administered within 24 h after the last missed intake of the COC. In some additional or other embodiments, the COC is resumed within 18 hours after the intake of ulipristal acetate, and even the same day as the intake of ulipristal acetate. In a further embodiment, an unprotected intercourse occurs within 120 hours before the intake of ulipristal acetate in the woman.

The present disclosure provides for a method for providing contraception to a woman. In one embodiment, the woman is using a combined oral contraceptive (COC) composition.

The method may comprise: (a) administering ulipristal acetate to the woman (i) after the woman has missed at least one daily dose of the COC composition, and (ii) within about 120 hours after the woman has unprotected intercourse in the woman's same menstrual cycle when the woman missed at least one daily dose of the COC composition; and (b) resuming administration of the COC composition to the woman within about 24 hours after administering ulipristal acetate.

The COC composition may comprise an estrogen and a progestogen. The estrogen may comprise ethinyl estradiol. The progestogen may comprise levonorgestrel.

In certain embodiments, the unprotected intercourse is within about 120 hours before or after the woman has missed at least one daily dose of the COC composition.

In certain embodiments, step (a) comprises administering orally about 30 mg ulipristal acetate to the woman.

In certain embodiments, the woman has missed no more than three consecutive daily doses of the COC composition. In certain embodiments, step (a) comprises administering ulipristal acetate to the woman within about 24 hours after the woman's last missed daily dose of the COC composition. In certain embodiments, step (b) comprises resuming administration of the COC composition within about 18 hours after administering ulipristal acetate.

In certain embodiments, the woman has used the COC composition for at least one menstrual cycle before the woman has missed at least one daily dose of the COC composition.

In certain embodiments, step (b) comprises resuming administration of the COC composition on a same day after administering ulipristal acetate.

In certain embodiments, step (b) comprises resuming administration of the COC composition within about 12 hours after administering ulipristal acetate.

In certain embodiments, step (a) comprises administering ulipristal acetate to the woman within about 72 hours after the woman has missed at least one daily dose of the COC composition.

The method may decrease the risk of ovulation in the woman's remaining menstrual cycle (e.g., compared with the risk of ovulation when ulipristal acetate is not administered). The method may inhibit or prevent ovulation in the woman's remaining menstrual cycle.

The present disclosure provides for a method for providing contraception to a woman. In one embodiment, the woman is using a combined hormonal contraceptive.

The method may comprise: (a) administering orally about 30 mg ulipristal acetate to the woman within about 120 hours after the woman has missed at least one daily dose of the combined hormonal contraceptive, and (b) resuming administration of the combined hormonal contraceptive within about 24 hours after administering ulipristal acetate.

The combined hormonal contraceptive may comprise an estrogen and a progestogen. The estrogen may comprise ethinyl estradiol. The progestogen may comprise levonorgestrel.

The combined hormonal contraceptive may be a combined oral contraceptive (COC) composition, a contraceptive patch, or a vaginal ring.

In certain embodiments, the woman has used the combined hormonal contraceptive (e.g., the COC composition) for at least one menstrual cycle.

In certain embodiments, the woman has missed no more than three consecutive daily doses of the combined hormonal contraceptive (e.g., the COC composition).

In certain embodiments, step (a) comprises administering ulipristal acetate within about 24 hours after the woman's last missed daily dose of the combined hormonal contraceptive (e.g., the COC composition).

In certain embodiments, step (b) comprises resuming administration of the combined hormonal contraceptive (e.g., the COC composition) within about 18 hours after administering ulipristal acetate.

In certain embodiments, step (b) comprises resuming administration of the combined hormonal contraceptive (e.g., the COC composition) to the woman on a same day after administering ulipristal acetate.

In certain embodiments, the woman has unprotected intercourse within about 120 hours before administering ulipristal acetate to the woman.

In certain embodiments, the woman has used the combined hormonal contraceptive (e.g., the COC composition) for at least one month.

### DETAILED DESCRIPTION OF THE INVENTION

Because ulipristal acetate acts as an antagonist of progesterone receptor, it is assumed that COCs, which contain a progestogen (namely an agonist of progesterone receptor), can negate or diminish the contraceptive effect of ulipristal acetate and vice versa. Consequently, the clinical guidelines are to wait at least 5 days after ulipristal acetate intake before initiating or resuming a hormonal combined contraceptive.

The Inventors have surprisingly found that women who use ulipristal acetate after missing COC pills should resume their COC as soon as possible. If they do not, they may be at risk of pregnancy if intercourse occurs during the remainder of their menstrual cycle.

A prospective, randomized, parallel-group clinical study was conducted to assess the impact of ulipristal acetate (30 mg) on the ovary function in women who misuse their combined oral contraceptive and are thus at higher risk of contraceptive failure. Details about this clinical trial are provided further below, in the Example section.

The women were divided into two groups: the first group were administered with EllaOne® (ulipristal acetate, 30 mg) after three missed pills and resumed the COC on the same day as EllaOne® intake. The second group waited for 5 days to resume the COC. Surprisingly, the Inventors showed that there is no interference between the COC and EllaOne®, when the COC is resumed at the same day as EllaOne®. Such an early restarting of the COC did not impair the efficacy of EllaOne® to prevent or delay ovulation resulting from the missed pills. Reciprocally, EllaOne® intake did not significantly impair the efficacy of the COC to maintain ovarian quiescence. Indeed, no ovulation at risk of pregnancy occurred in the group resuming COC intake on the same day as Ella® intake, at any time of the cycle. In contrast, 17.4% of the women who delayed the intake of COC ovulated later in the menstrual cycle and would have been thus at risk of unintended pregnancy if an intercourse had occurred. Noteworthy, the ovarian activity was more important in the women who delayed the intake of COC as compared to women who resumed COC on the same day as ulipristal acetate intake.

The present invention thus offers a reliable and safeguard method of contraception for women who experiment a suspected failure of their hormonal combined contraceptive.

Thus, the invention relates to a method for providing contraception in a woman comprising administering ulipristal acetate or a metabolite thereof within about 120 hours after a suspected failure of a hormonal combined contraceptive and resuming the hormonal combined contraceptive within about 24 hours after the intake of ulipristal acetate or a metabolite thereof in said woman.

The invention also relates to a method for reducing the likelihood of ovulation in a woman experimented a suspected failure of her hormonal combined contraceptive, wherein the method comprises administering ulipristal acetate or a metabolite thereof within 120 hours after said suspected failure and resuming the regular hormonal combined contraceptive within 24 hours after the intake of ulipristal acetate or the metabolite thereof in said woman.

Typically, the suspected failure of the contraceptive is due to a misuse of said hormonal combined contraceptive in the woman.

The woman generally has an intercourse within the menstrual cycle in which the misuse of the contraceptive occurs, preferably close to said misuse.

Accordingly, the invention relates to a method for providing contraception in a woman, wherein:
the woman is under hormonal combined contraception but experiments a suspected failure of said hormonal combined contraceptive due to a misuse and
wherein the method comprises administering ulipristal acetate or a metabolite thereof after the misuse and within 120 h after an unprotected intercourse occurring in the same menstrual cycle as the misuse and resuming the hormonal combined contraceptive within about 24 hours after the intake of ulipristal acetate or a metabolite thereof in said woman.

The conditions “after the misuse” and “within 120 h after an unprotected intercourse” are cumulative which means that, in said embodiment of the invention, the administration of ulipristal acetate or a metabolite thereof in the woman is both after the misuse and within 120 hours after the unprotected intercourse in the woman.

As illustrated in the example, the method of the invention enables to inhibit ovulation in the remaining part of the menstrual cycle following the intake of ulipristal acetate in the woman. In other, the invention prevents the woman from getting pregnant following an intercourse occurring within 120 hours before the intake of ulipristal acetate but also following an intercourse occurring later in the menstrual cycle.

Accordingly, in a more general aspect, the invention also relates to a method for providing contraception in a woman comprising administering ulipristal acetate or a metabolite thereof within about 120 hours after at least one misuse of a hormonal combined contraceptive and resuming the hormonal combined contraceptive within about 24 hours after the intake of ulipristal acetate or a metabolite thereof in said woman.

In such an embodiment, the woman may have an unprotected intercourse within 120 hours before the intake of ulipristal acetate or later in the same menstrual cycle.

More generally, the invention further relates to a method for providing contraception in a woman, wherein the woman is subjected to hormonal combined contraceptive but experiments at least one misuse of said hormonal combined contraceptive and wherein the method comprises administering ulipristal acetate or a metabolite thereof within 120 hours after said at least one misuse and resuming the hormonal combined contraceptive within 24 hours after the intake of ulipristal acetate or a metabolite thereof in said woman.

Another object of the invention is a method for inhibiting ovulation in a woman under hormonal combined contraception, after a misuse of said contraception, the method comprises administering ulipristal acetate or a metabolite thereof within 120 hours after said misuse of contraception and resuming the hormonal combined contraception within 24 hours after the intake of ulipristal acetate or a metabolite thereof in said woman, whereby the ovulation is inhibited in the remaining part of the menstrual cycle after said misuse or failure.

A further object of the invention is a method for providing contraception in a woman at risk of an unintended pregnancy due to a misuse of her regular hormonal combined contraceptive, wherein ulipristal acetate or a metabolite thereof is administered to the woman within 120 hours after said misuse and the hormonal combined contraception is resumed within 24 hours after the intake of ulipristal acetate or a metabolite thereof in said woman.

In a particular aspect, the invention relates to a method for providing contraception in a woman, wherein:
the woman is daily administered with a combined oral contraceptive (COC) and misses at least one daily intake of said combined oral contraceptive, and
the method comprises orally administering an amount of 30 mg of ulipristal acetate within 120 hours after the missed intake of the COC and resuming the intake of the COC within 24 hours after the intake of ulipristal acetate in said woman, whereby the ovulation is inhibited for the remaining period of the menstrual cycle following the missed intake of the COC.

In another aspect, the invention relates to a method for providing contraception in a woman, wherein:
the woman is daily administered with a combined oral contraceptive (COC) and misses at least one daily intake of said combined oral contraceptive, and
the method comprises orally administering an amount of 30 mg of ulipristal acetate after said at least one missed intake of the COC and within 120 hours after an unprotected intercourse occurring the same menstrual cycle as the missed intake of the COC and resuming the intake of the COC within 24 hours after the intake of ulipristal acetate in said woman, whereby the ovulation is inhibited for the remaining period of the menstrual cycle following the missed intake of the COC.

In a further aspect, the Invention also relates to method for maintaining ovarian quiescence after at least one missed intake of a combined oral contraceptive (COC) in a woman, which comprises administering ulipristal acetate within 120 hours, preferably within 24 hours, after the at least one missed intake of the COC and resuming the COC within 24 hours after the intake of ulipristal acetate, in said woman. In some embodiments, the misuse is at most three consecutive missed intakes of the COC. Ulipristal acetate is typically administered by oral route, in an amount of 30 mg in the woman.

The Woman (or Female Subject)

The woman is of childbearing age, typically between 14 and 40 years old, preferably between 18 and 40 years old, e.g. between 18 and 30 years old.

Said woman uses the hormonal combined contraceptive as a regular birth control method, in order to avoid pregnancy. In the context of the invention, said woman is at risk of an unintended pregnancy due to a suspected failure of her hormonal combined contraceptive, as explained below.

As used herein, “a suspected failure of a hormonal combined contraceptive” means that the contraceptive treatment taken by the women may fail to protect the woman from pregnancy. Preferably, “a suspected failure of a hormonal combined contraceptive” refers to an increase in the likelihood to have an ovulation in a menstrual cycle of the woman, if no safeguard measure is taken. Typically, the suspected failure of the hormonal combined contraceptive results from a misuse of said contraceptive in the woman, as explained further below.

As used herein, “a woman is at risk of an unintended pregnancy” means that the woman is likely to ovulate, namely having an ovulation, in the menstrual cycle wherein she experiments the suspected failure of contraception.

In the context of the Invention, the term “ovulation” refers to an ovulation at risk of pregnancy, namely to the release of an oocyte from the ovaries in the woman in the context of a hormonal environment which favorable to pregnancy if an intercourse occurs in the fertile window, namely to a hormonal environment favorable to fertilization of the egg and implantation of embryo.

In other words, the ovulation is at risk of pregnancy if a pregnancy may result from an intercourse during the fertile window in the woman. The fertile window in a woman is generally defined as beginning 5 days before ovulation and ending 24 hours after ovulation. In some embodiments, the ovulation is at risk of pregnancy if the plasma level of estradiol is above 0.1 nmol/1 and the plasma level of progesterone is above 10 nmol/1 after the ovulation.

In some embodiments, the woman has an intercourse occurring close to the suspected failure of contraception, e.g. within 7 days, e.g. within about 120 hours, about 72 hours, about 48 hours or about 24 hours, before and/or after the suspected failure of contraception.

Hormonal Combined Contraceptives and Misuses Thereof

In the context of the invention, hormonal contraception refers to birth control methods that act on the endocrine system and relies on the release of steroidal hormones or synthetic analogues thereof to exert contraceptive effect in the woman. There are two main types of hormonal contraceptive products: hormonal combined contraceptive products which contain both an estrogen and a progestogen, and progestogen-only contraceptive product which contains only progesterone or one of its synthetic analogues (namely a progestogen, also called progestin). Hormonal combined contraceptives mostly act by inhibiting ovulation in the woman by establishing ovarian quiescence.

As used herein, a hormonal combined contraceptive refers to a contraceptive product releasing a progestogen and an estrogen as contraceptive agents. The route of administration can be oral, transdermal, intrauterine, subcutaneous, intramuscular, or intravaginal.

The hormonal combined contraceptives typically contain ethinyl estradiol or estradiol as estrogen. A plurality of progestogens can be used in hormonal combined contraceptive products. Progestogens encompass, without being limited to, norethindrone, norethindrone acetate, ethynodiol diacetate, dydrogesterone, medroxy-progesterone acetate, norethynodrel, allylestrenol, lynoestrenol, medrogestone, norgestrienone, dimethiderome, ethisterone, cyproterone acetate, levonorgestrel, gestodene, desogestrel, etonogestrel, norgestimate, nestorone, dienogest, norelgestromin and drospirenone.

In some embodiments, the hormonal combined contraceptive comprises ethinyl estradiol as estrogen and a progestogen selected from the group consisting of levonorgestrel, norgestrel, norelgestromin, drospirenone, norgestimate, desogestrel, etonogestrel, norethindrone acetate and norethindrone.

Hormonal combined contraceptives generally refer to regular contraceptives, namely to contraceptives that are intended to be used over several weeks and even several months to provide a sustained contraceptive effect.

Hormonal combined contraceptives encompass, without being limited to, combined oral contraceptives (“COCs”, also referred to “combined oral contraceptive pill (COCP)” or the birth control pills) and medical devices able to deliver both estrogen and progestogen such as contraceptive transdermal patch, hormonal intrauterine device and vaginal ring.

Hormonal combined contraceptives also encompass subcutaneous implants and intramuscular, injectable suspensions which forms prolonged-release depot. Such subcutaneous implants and intramuscular depot are able to release contraceptive daily amounts of progestogen and estrogen during several consecutive weeks or months.

Contraceptive patches are designed to be applied on an intact area of the skin and to enable the continuous administration of the contraceptive agents through the skin. A treatment cycle with a contraceptive patch lasts 4 weeks. Typically, a patch is leaved on the skin during 7 days. Every 7 days during three weeks, the patch is removed and a new one is applied. Then, no patch is generally not applied during 7 days (hormone-free period) in order to allow withdrawal bleedings. At the end of the hormone-free period, a new treatment cycle is restarted by applying a new patch on the skin. The patch may comprise ethinyl estradiol as estrogen and norelgestromin as progestogen. The patch is able to release a daily amount of estrogen and progestogen sufficient to provide contraception, for instance from about 5 to 50 μg, typically about 34 μg of ethinyl estradiol and from about 50 to 300 μg, e.g. about 200 μg of norelgestromin per 24 hours.

Vaginal rings are generally designed to be inserted in the vagina of the woman and release the contraceptive agents during 21 days. Typically, the vaginal ring is inserted and leaved in the vagina during 21 days. At day 21, the vaginal ring is removed. Generally no vaginal ring is used during the following 7 days (hormone-free period) in order to allow withdrawal bleedings. At the end of the hormone-free period, a new treatment cycle is restarted by replacing a new vaginal ring.

The vaginal ring is able to release a daily amount of estrogen and progestogen sufficient to provide contraception, for instance from about 5 to 50 μg, typically about 15 μg of ethinylestradiol and from about 80 to 300 μg, e.g. about 120 μg of etonogestrel per 24 hours.

As mentioned above, Combined Oral Contraceptive pills (COC or COCP) comprise both a progestogen and an estrogen. These contraceptive pills are generally designed to be taken every day, preferably at regular time. There are many different formulations or brands. A typical pack contains pills for daily intake over a 28-day period, or cycle, repeated over several months. For the first 21 days of the cycle, the woman generally takes a daily pill that contains the estrogen and the progestogen. The last 7 days of the cycle are hormone free days. Some packs only contain 21 pills and the woman is then advised to take no pill for the following week. Other packs contain 7 additional placebo pills, whereby the woman takes a pill every day without any break between two consecutive pack intakes. Some other COC relies on a regimen based on the administration of active pills during 24 days or 26 days following by the administration of daily placebo pills during 4 days or 2 days. Other COCs rely on the daily intake of active pills during 84 followed by 7 days of placebo pills. Alternatively, the placebo pills can be replaced by pills containing a non-contraceptive amount of a progestogen. The progestogen can be the same as or different from that used in the active pills.

The active combined oral pills can be continuously administered to the woman, e.g. daily administered to the woman without any hormone free period in order to avoid withdrawal bleeding. The daily dosage of the estrogen in the COC is typically from 5 μg to 100 μg, preferably from 10 to 60 μg or from 10 μg to 40 μg. The daily dosage of the progestogen in the COC depends on the progestogen used, namely its strength as an agonist of the progesterone receptor. Typically the dosage of the progestogen in the daily active COC pill ranges from 0.01 mg to 5 mg, typically from 0.05 mg to 4 mg. For instance, drospirenone is generally present at a daily dosage of 1 mg to 4 mg, typically 3 mg. Levonorgestrel may be present in the COC pill at a daily dosage of about 0.05 to 0.3 mg, e.g. from 0.1 to 0.2 mg.

There are a plurality of hormonal combined contraceptives available on the market. For instance, one can cite:
Nuvaring® which is a vaginal ring comprises ethinyl estradiol and etonogestrel
Evra® which is a contraceptive transdermal patch comprising ethinyl estradiol as estrogen and norelgestromin as progestogen, and
COCs such as Yasmin® or Yaz® (ethinyl estradiol/drospirenone), Vienva® (ethinyl estradiol/levonorgestrel), Ortho Tri-Cyclen (ethinyl estradiol/noregestimate), Loestrin FE 1/20® (ethinyl estradiol/norethindrone acetate), Kariva® (ethinyl estradiol/desogestrel), Nortrel 28® (ethinyl estradiol/norethindrone), Valette® (ethinyl estradiol/dienogest), and Gynera® (ethinyl estradiol/gestodene).

In the context of the invention, the hormonal combined contraceptive is administered to the woman to provide regular contraception. In other words, the woman uses the hormonal combined contraceptive as a regular birth control method to avoid pregnancy. Typically, the woman has been using the hormonal combined contraceptive for at least one menstrual cycle or month such as for at least 2, 3, 4, 5, 6, 9, 10, 11 menstrual cycles or months and even for at least 1 year, e.g. for at least 2, 3, 4 or 5 years.

In the context of the invention, the woman experiments a suspected failure of her hormonal combined contraceptive which means that the woman is at risk of an intended pregnancy even if she is under a regular contraception.

Typically, a suspected failure of the hormonal combined contraceptive refers to, or results from, a misuse of said contraceptive.

As used herein, a misuse use of a combined hormonal contraceptive typically refers to an inconsistent or incorrect use of the contraceptive, more generally to a non-compliance with the dosage regimen specified in the labelling of the hormonal combined contraception. Such a misuse may expose the woman to a risk of ovulation and thus to a risk of unintended pregnancy. A misuse of the hormonal contraceptive can also encompass deficiency in the contraceptive product (e.g. the fact that a contraceptive patch abnormally comes off or a vaginal ring is abnormally dislodged from the vagina) and exposure to conditions or disorders preventing an appropriate use of the contraceptive product. In the context of COCs, such conditions or disorders encompass nausea, diarrhea, and the intake of drugs or substances which can impair the contraceptive effects of estrogen and/or progestogens. As a further example, such conditions encompass excessive sweating resulting in a loss of adherence, and even the removal, of the contraceptive patch.

In some embodiments, the misuse is an incorrect use of the hormonal combined contraceptive, preferable a use non-compliant with the labelling of the contraceptive.

As used herein, the term “misuse” encompasses intentional or unintentional, incorrect, use of the hormonal combined contraceptive. Preferably, the misuse refers to an unintentional, incorrect use of the hormonal combined contraceptive in the woman. In the context of a combined oral contraceptive, a typical unintentional, incorrect use is the fact that the woman misses or forgets to take at least one daily pill of said COC.

The type of misuse depends on the hormonal combined contraceptive used by the woman.

When the hormonal combined contraceptive is a COC, the misuse can be, for example, missing at least one (e.g. 1, 2, 3, 4, 5 or more) daily intake of the COC, taking a placebo pill instead of an active pill or vomiting.

As explained above, COC is generally based on daily intakes at about the same time of the day.

As used herein, “a missed pill” or “a missed intake of the COC or the pill” means that the woman has missed a daily intake of her pill and is more than 12 hours preferably more than 24 hours late for taking the pill.

As used herein, the woman has missed 2 or more consecutive pills means that she is more than 48 h late for taking the pill.

In some embodiments, the misuse refers to missing at least one daily intake (e.g. 1, 2 or 3) during the first seven days of the cycle.

In a particular embodiment, the failure refers to miss three consecutive daily intakes of the pills during the first week of the cycle.

For instance, a misuse may be the fact that the woman has forgotten to take at least one pill of her COC and is more than 12 hours late taking the pill.

In some embodiments, the woman has missed one daily intake of the COC and is more than 12 hours, preferably more than 24 hours and up to 48 hours late for taking the pill.

When the hormonal combined contraceptive is a vaginal ring, the misuse can be, for example, the expulsion of the vaginal ring (without replacing it within 3 hours after the expulsion) or a delay in replacing the vaginal ring.

When the hormonal combined contraceptive is a transdermal patch, the misuse typically occurs when the patch comes off partially or completely during the use period, is early removed, is not changed in due time or is not applied on the skin in due time after the free hormone period.

When the hormonal combined contraceptive is a subcutaneous implant or a intramuscular depot injection, a misuse is for instance missing to replace the implant or to repeat the injection in the timing determined by the labelling. The woman is more than 7 days late for replacing a new implant or for a new depot injection.

In a particular aspect of the invention, the hormonal combined contraceptive is a combined oral contraceptive and the misuse is at least one missed pill in a menstrual cycle of the woman.

Ulipristal Acetate or a Metabolite Thereof

Ulipristal acetate, formerly known as CDB-2914, is 17α-acetoxy-11β-[4-N,N-dimethylamino-phenyl]-19-norpregna-4,9-diene-3,20-dione, represented by formula I:

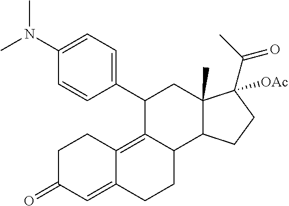 [SMILES: CC(=O)O[C@]1(C(C)=O)CCC2C3CCC4=CC(=O)CCC4=C3C(c3ccc(N(C)C)cc3)C[C@@]21C]

This compound, and methods for its preparation, are described in U.S. Pat. Nos. 4,954,490, 5,073,548, and 5,929,262, and international patent applications WO2004/065405 and WO2004/078709. Metabolites of CDB-2914, include those described in Attardi et al, Journal of Steroid Biochemistry & Molecular Biology, 2004, 88: 277-288, e.g. monodemethylated CDB-2914 (CDB-3877); didemethylated CDB-2914 (CDB-3963); 17alpha-hydroxy CDB-2914 (CDB-3236); aromatic A-ring derivative of CDB-2914 (CDB-4183).

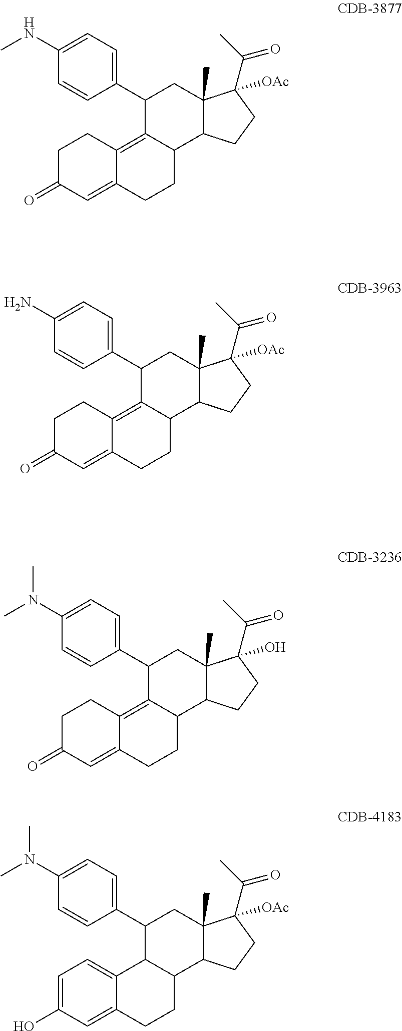 [SMILES: CNc1ccc(C2C[C@@]3(C)C(CC[C@]3(OC(C)=O)C(C)=O)C3CCC4=CC(=O)CCC4=C23)cc1 ; CC(=O)O[C@]1(C(C)=O)CCC2C3CCC4=CC(=O)CCC4=C3C(c3ccc(N)cc3)C[C@@]21C ; CC(=O)[C@@]1(O)CCC2C3CCC4=CC(=O)CCC4=C3C(c3ccc(N(C)C)cc3)C[C@@]21C ; CC(=O)O[C@]1(C(C)=O)CCC2C3CCc4cc(O)ccc4C3C(c3ccc(N(C)C)cc3)C[C@@]21C]

Preferred metabolites are 17α-acetoxy-11β-[4-N-methylamino-phenyl]-19-norpregna-4,9-diene-3,20-dione (CDB-3877) and 17α-acetoxy-11β-[4-aminophenyl]-19-norpregna-4,9-diene-3,20-dione (CDB-3963).

In a more preferred embodiment, the methods of the invention are implemented with ulipristal acetate.

Ulipristal acetate or a metabolite thereof can be administered by various routes, including buccal, sublingual, parenteral, transdermal, vaginal, or rectal route. Any type of pharmaceutical forms containing ulipristal acetate or a metabolite thereof may be used for implementing the contraceptive method of the invention. Such forms encompass suppositories, injectable solutions, syrups, creams, transdermal patches, transdermal spray, capsules, tablets, vaginal tablets, mucoadhesive tablets, powders, suspensions, granules, and the like.

In a preferred embodiment, ulipristal acetate or a metabolite thereof is administered by oral route. In that case, any kind of oral form can be used such as liquid forms, including suspension or syrup, and solid forms, such as a coated or uncoated tablets and capsules.

Preferably, the oral form used to administer ulipristal acetate or a metabolite thereof is an immediate-release oral form, such as an immediate-release tablet.

Ulipristal acetate or a metabolite thereof may be formulated according to standard methods as described in Remington: The Science and Practice of Pharmacy (Lippincott Williams & Wilkins; Twenty first Edition, 2005). Pharmaceutically acceptable excipients that may be used to formulate the contraceptive compositions of the invention are, among others, described in the Handbook of Pharmaceuticals Excipients, American Pharmaceutical Association (Pharmaceutical Press; 6th Revised edition, 2009).

Examples of appropriate excipients include, but are not limited to, fillers, carriers, diluents, binders, anti-caking agents, plasticizers, disintegrants, lubricants, flavors, buffering agents, stabilizers, colorants, dyes, anti-oxidants, anti-adherents, softeners, preservatives, surfactants and glidants.

In some embodiments, the contraceptive composition comprises one or more excipients selected from the group of binders, diluents, disintegrants, glidants and lubricants.

Examples of diluents include, without being limited to, microcrystalline cellulose, starch, modified starch, dibasic calcium phosphate dihydrate, calcium sulfate trihydrate, calcium sulfate dihydrate, calcium carbonate, mono- or disaccharides such as lactose, dextrose, sucrose, mannitol, galactose and sorbitol, xylitol and combinations thereof.

Examples of binders include, without being limited to, starches, e.g., potato starch, wheat starch, corn starch; gums, such as gum tragacanth, acacia gum and gelatin; hydroxypropyl cellulose, hydroxyethyl cellulose, hydroxypropyl methyl cellulose; polyvinyl pyrrolidone, copovidone, polyethylene glycol and combinations thereof.

Examples of lubricants include, without being limited to, fatty acids and derivatives thereof such as calcium stearate, glyceryl monostearate, glyceryle palmitostearate magnesium stearate, zinc stearate, or stearic acid, or polyalkyleneglycols such as PEG.

The glidant may be selected among colloidal silica, dioxide silicon, talc and the like.

Examples of disintegrants encompass, without being limited to, crospovidone, croscarmellose salts such as sodium croscarmellose, starches and derivatives thereof.

Examples of surfactants encompass, without being limited to, simethicone, triethanolamine, les polysorbate and derivatives thereof such as Tween® 20 or Tween® 40, poloxamers, fatty alcohol such as laurylic alcohol, cetylic alcohol and alkylsulfate such as sodium dodecylsulfate (SDS).

The pharmaceutical composition may be obtained merely by mixing ulipristal acetate or a metabolite thereof with one or several excipients and may be shaped by standard methods. For instance, a tablet may be obtained by wet granulation, dry granulation or direct compression.

Ulipristal acetate or a metabolite thereof may be micronized, co-micronized with a suitable excipient, e.g. a surfactant such as SDS, or provided as a solid dispersion.

Examples of appropriate oral compositions to administer ulipristal acetate or a metabolite thereof are provided in WO2010/066749, WO2014/072646, WO2014/072647 and WO2014/087106.

In a particular aspect, ulipristal acetate or a metabolite thereof is orally administered to the woman in the form of an uncoated or coated tablet, e.g. as described in WO2010/066749.

Typically Ella® or EllaOne® tablets can be used to implement the invention.

Ulipristal acetate is administered in an amount effective to thwart the risk of ovulation resulting from the misuse of the hormonal combined contraceptive in the woman. Typically, ulipristal acetate is administered at an amount of 20 mg to 50 mg, preferably from 25 to 35 mg, and more preferably at about 30 mg. Such an amount can be administered in the form of one or several unit dosages.

The Regimen

In a certain aspect, ulipristal acetate or a metabolite thereof is administered within 120 hours after a suspected failure of contraception.

In some aspects, ulipristal acetate or a metabolite thereof is administered after a misuse of the hormonal combined contraceptive and within 120 hours after an unprotected intercourse occurring in the same menstrual cycle as the misuse.

In the context of the present invention, the conditions “after a misuse of the hormonal combined contraceptive” and “within 120 hours after an unprotected intercourse” are cumulative, which means that the administration of ulipristal acetate or a metabolite thereof is both after said misuse and within 120 hours after the unprotected intercourse in the woman. As used herein, the menstrual cycle of the women refers to a treatment cycle of the hormonal combined contraceptive and generally lasts 28 days. The treatment cycle of hormonal combined contraceptives of interest are depicted further above.

Preferably, the unprotected intercourse is close to the misuse, e.g. typically occurs within 120 h, such as within 72 h, 48 h, 24 h, or 12 h, before and/or after the misuse of the hormonal combined contraceptive.

As used herein, “an unprotected intercourse” refers to an intercourse wherein no contraceptive barrier such as condom has been used.

In some other aspects, ulipristal acetate or a metabolite thereof is administered within 120 hours after the at least one misuse of the hormonal combined contraceptive, e.g. within 96 hours, 72 hours, 48 hours or 24 hours after the at least one misuse of the hormonal combined contraceptive.

Preferably, the administration of ulipristal acetate or a metabolite thereof is performed by oral route.

Preferably, one single administration of ulipristal acetate is performed in the woman.

As illustrated in the experimental section, resuming the hormonal combined contraceptive should not be excessively delayed after the intake of ulipristal acetate or a metabolite thereof. In the context of the invention, the hormonal combined contraceptive is resumed within 24 hours after the intake of ulipristal acetate or a metabolite thereof, e.g. within 18 hours, 12 hours, 6 hours and even 1 hour after the intake of ulipristal acetate or a metabolite thereof.

In some embodiments, ulipristal acetate or a metabolite thereof is orally administered within 48 hours after the misuse of the hormonal combined contraceptive and the hormonal combined contraceptive is resumed within 18 hours after the intake of ulipristal acetate.

In a particular embodiment, the hormonal combined contraceptive is resumed on the same day as the ulipristal acetate or a metabolite thereof.

When the hormonal combined contraceptive is a medical device such as a patch or a vaginal ring, resuming the hormonal combined contraceptive may encompass replacing the used medical device in an appropriate part of the body of the woman or applying a new, non-used, identical medical device to the woman. In a preferred embodiment, a new medical device is applied to the woman.

When the hormonal combined contraceptive is a contraceptive oral one, resuming the contraceptive refers to restart the daily intake of the contraceptive from the pill which directly follows the missed or not properly used pill(s) in the pack.

### A Particular Embodiment According to the Invention

In a particular embodiment of the invention, the hormonal combined contraceptive taken by the woman is a combined oral contraceptive. The combined oral contraceptive is as described above. Said contraceptive preferably comprises ethinyl estradiol as estrogen. The daily dosage of ethinyl estradiol in the COC is generally from 5 μg to 100 μg, such as from 10 μg to 50 μg.

The progestogen may be selected from the group consisting of levonorgestrel, norgestrel, drospirenone, norgestimate, desogestrel, norethindrone acetate and norethindrone. The daily dosage of the progestogen is typically from 0.01 mg to 5 mg.

A preferred progestogen is levonorgestrel.

The combined oral contraceptive is based on a 28-day cycle which is repeated overtime. The 28-day cycle comprises a first period of 21 days wherein the woman takes a daily pill containing the estrogen and the progestogen followed by a 7-day free-hormone period wherein the woman takes no pill or a daily placebo.

In some embodiments, the active pills of the COC comprises:
ethinylestradiol at a daily dosage from 10 μg to 50 μg, e.g. from 20 to 40 μg and
levonorgestrel at a daily dosage from 0.1 mg to 0.3 mg, e.g. from 0.1 mg to 0.2 mg.

The woman has been taking said hormonal combined contraceptive for at least one menstrual cycle (e.g. for at least 2, 3, or 4 menstrual cycles) when the misuse occurs.

The misuse is at least one missed pill, which means that one or several consecutive pills can be missed. Preferably the misuse refers to at most three consecutive missed pills (i.e. 1, 2 or 3 pills are missed). It goes without saying that said misuse occurs within the first period of 21 days of the COC regimen. In some embodiments, said misuse occurs within the 7 first days of the cycle.

In a particular embodiment, the woman misses at least one pill (1, 2 or 3 pills) within the first 7 days of the COC treatment cycle.

The woman is administered with ulipristal acetate by oral route at a dosage of 30 mg.

In some embodiments, said administration occurs within 120 hours after the misuse, preferably within 48 or 24 hours after the misuse. In some embodiments, an unprotected intercourse may occur within 120 hours before the intake of ulipristal acetate.

In other embodiments, said administration occurs after the misuse and within 120 hours after an unprotected intercourse occurring in the same menstrual cycle in the woman. In such a case the conditions “after misuse” and “within 120 hours after the intercourse” are cumulative, which means that the timing of the administration of ulipristal acetate meets the two conditions. Preferably, one single, oral, administration of ulipristal acetate is performed. Preferably, said intercourse is close to the misuse, e.g. within 120 hours, 72 hours, 48 hours or 24 hours after or before the misuse. In a particular embodiment, said administration of ulipristal acetate is performed within 120 hours after an unprotected intercourse and within 24 hours after the at least one misuse of the COC.

The hormonal contraceptive pill is resumed within 24 hours after the intake of ulipristal acetate, preferably within 12 hours, 6 hours and even 1 hours after the intake of ulipristal acetate. In some embodiments, the hormonal contraceptive pill is resumed on the time of day at which the woman used to take it.

### Example: A Prospective, Randomized, Parallel-Group Study to Assess the Effects on Ovarian Activity of ellaOne® (Ulipristal Acetate 30 mg Single Dose) Taken after Three Consecutive Days of Missed Combined Oral Contraceptive Pills

Objective:

To determine the effect on ovarian activity and ovulation, of waiting five days before re-starting combined oral contraception (COC), or restarting immediately, having taken ulipristal acetate (UPA) for emergency contraception (EC) after missing three COC pills.

Study Design:

Women were enrolled for three cycles of COC use in a 21/7 regimen. In cycle 3 all women omitted COC pills for three consecutive days (days 5, 6, 7) and on day 8 took UPA-EC (30 mg). Half the women restarted their COC pills that same day while the remainder waiting for five days before restarting. Transvaginal ultrasound (TVUS) and blood sampling for estrogen and progesterone (E and P) was undertaken in all women on days 4, 8, 11, 13, 15, 18, 22 and 26. If a follicle of >13 mm diameter was seen, TVUS and blood sampling were performed daily until a post-ovulatory follicle was seen.

### Detailed Protocol:

Women aged 18-30, weighing ≤30 kg/m² participated in the study during use of three consecutive packets of COCP. Women with contraindications to the COC or with conditions associated with anovulation (e.g. polycystic ovarian syndrome), and women who were breastfeeding or using any other hormonal contraception or an intrauterine device (IUD) were excluded. Screening was undertaken during cycle 1. Throughout the study women kept a daily diary of pill intake and vaginal bleeding. All women were instructed to take their pills at the same time every evening and to follow standard rules during the first two cycles of pill use for missed pills or for vomiting after taking a pill. The duration of COC intake in cycle 1 was adjusted (to no less than 14 and no more than 21 pills) to allow the first day of cycle 2 (baseline cycle—BSL) to fall on a weekday. After a seven-day hormone free interval (HFI) all women switched to the study COC containing 30 μg ethinyl estradiol/150 μg levonorgestrel with seven placebo tablets. Cycle three (Experimental cycle—EXP), started after a seven-day HFI at the end of BSL. After transvaginal ultrasound (TVUS) on day 1 of pill intake women were randomized so that an equal number of women with ovarian follicles of diameter ≤10 mm, or >10 mm and ≤13 mm or >13 mm were allocated to one of two study arms. After 4 days of COC, all women deliberately missed their COC pill on three consecutive days (5, 6, 7) and on the morning of day 8 all took a single 30 mg tablet of ellaOne® (ulipristal acetate 30 mg). Women in arm 1 of the study (immediate re-start) resumed daily COC intake that evening (D8) while those in arm 2 waited 5 days, re-starting their OC pill on the evening of D13 (delayed re-start). Once restarted, the COCP was continued until the packet was finished. TVUS and blood sampling were performed as shown in FIG. 1. A high sensitivity urinary pregnancy test was undertaken on day 1 of BSL and EXP and at the final study visit one or two days after the HFI following the end of the pill pack. Adverse event information was collected at every visit and blood sampling for haemoglobin and liver function was performed four times during the study.

TABLE 1
Timing of transvaginal ultrasound and blood
sampling in baseline and experimental cycles

| | Experimental cycle (EXP) |
|---|---|
| Baseline cycle | COC pills missed on days 5, 6, 7; UPA-EC taken D8 |
| (BSL) | COC restarted on day 8 (immediate start) or day 13 (delayed start) |
| Days 1, 4, 8, 15, 22 | Days 1, 4, 8, 11, 13, 15, 18, 22, 26 |
| Transvaginal | Transvaginal ultrasound (TVUS) and blood sampling |
| ultrasound (TVUS) | for estrogen (E) & progesterone (P) |
| and blood sampling | If follicle diameter > 13 mm was seen daily TVUS and blood |
| for estrogen (E) and | sampling performed until a clear postovulatory image was seen or |
| progesterone (P) | until E2 was ≤ 0.1 nmol/L, following which TVUS continued |
| | every second day for six more days. |

### Statistical Analysis

An ovarian activity score developed from the Hoogland score (Hoogland and Skouby 1993, Contraception, 1993. 47(6): p. 583-90). The ovary was considered quiescent (OSq) if estradiol concentration was ≤0.1 nmol/L and progesterone ≤5 nmol/L even if a follicle of >13 mm was present. The presence of a follicle >13 mm with estradiol ≤0.1 nmol/L and progesterone ≤10 nmol/L or if TVUS suggested collapse of a large follicle but with progesterone levels consistently <10 nmol/L or between 10 and 30 nmol/L on only one sampling occasion was defined as ovarian activity not at risk of pregnancy (OSa). Ovulation was considered at risk of pregnancy (OSp) if there was a postovulatory image on TVUS and progesterone concentrations were >30 nmol/L on one occasion or between 10-30 nmol/L on two consecutive occasions.

The proportion of women who were at risk of pregnancy (OSp) at any time during the experimental cycle, including those who ovulated within the five days following ellaOne® intake, was estimated and the exact (Clopper-Pearson) two-sided 95% confidence interval was provided for each arm. The difference between the two arms was tested through an exact without stratum adjustment due to insufficient number of women in the self-weighted strata. A generalized estimating equation was used to estimate the parameters of a generalized linear model based on binomial distribution for modeling binary variables such as a given ovarian activity status. A mixed model for repeated measures was fitted for quantitative response such follicle size. Kaplan Meier approach and log rank test were used for time to event analyses.

### Results:

In total 74 women were screened and 54 were randomized 27 women were randomized to Arm 1 of the study (immediate COC pill re-start), 26 of whom completed the study. 27 women were randomized to Arm 2 (delayed COC pill re-start), 23 completed the study. Women were of mean age 25±3.7 years, only one was not white (in Arm 1), mean weight was 64.2±8.5 kg and mean BMI 23.0±3.0 kg/m². There were no meaningful differences between the two arms of the study in respect of demographic characteristics. Two women were sexually inactive throughout the study, 63 agreed to abstain for sexual intercourse between days 5 and 19 of the experimental cycles.

No ovulation, with or without risk of pregnancy, was reported for any woman at any time during the baseline cycle. During the baseline cycle, most women were in ovarian quiescence on most days when monitored. During the baseline cycle, the risk of OSa was not different between the arms (OR 1.14 [0.24; 5.49], p=0.873).

In contrast, the pattern of ovarian activity was dramatically distinct between the two arms during the experimental cycle, as shown in Table 2. Arm 1: COC resumed on the day of ellaOne® intake (N=26)

TABLE 2
Time course of ovarian activity during the experimental cycle for women in both arms of the study.

| | | D 1 | D 4 | D 8 | D 11 | D 13 | D 15 | D 18 | D 22 | D 26 |
|---|---|---|---|---|---|---|---|---|---|---|
| Experimental | OSq | 26/26 | 25/26 | 24/26 | 25/26 | 25/26 | 25/26 | 25/26 | 25/26 | 24/25 |
| Arm 1 | OSa | 0/26 | 1/26 | 2/26 | 1/26 | 1/26 | 1/26 | 1/26 | 1/26 | 1/26 |
| Immediate | OSp | 0/26 | 0/26 | 0/26 | 0/26 | 0/26 | 0/26 | 0/26 | 0/26 | 0/26 |
| COCP re- | | | | | | | | | | |
| start | | | | | | | | | | |
| Arm 2 | OSq | 22/23 | 20/23 | 19/23 | 17/23 | 17/22 | 16/23 | 14/23 | 17/23 | 15/23 |
| Delayed | OSa | 1/23 | 3/23 | 4/23 | 6/23 | 5/22 | 7/23 | 8/23 | 2/23 | 4/23 |
| COCP | OSp | 0/23 | 0/23 | 0/23 | 0/23 | 0/22 | 0/23 | 1/23 | 4/23 | 4/23 |
| re-start | | | | | | | | | | |

Arm 1: COC resumed on the day of ellaOne ® intake (N = 26)
Arm 2: COC resumed five days after ellaOne ® intake (N = 23)

Noteworthy, no woman who resumed the COC immediately after taking Ellaone® ovulated with risk of pregnancy, at any time of the experimental cycle. However, one woman showed evidence of an ovarian activity but without a risk of pregnancy (Osa): This woman showed evidence of ovarian activity on days 4 and 8 of the experimental cycle, had a postovulatory follicle on TVUS on day 13 but with progesterone concentrations maintained at <10 nmol/L throughout the remainder of the cycle.

The situation was different in Arm 2. Eight women who delayed re-starting their pill showed evidence of the resumption of follicle growth during the remainder of the experimental cycle. Four women (17.4% CI 5.0-38.8) who delayed re-starting their COC pill for five days ovulated with risk of pregnancy (postovulatory follicles with normal ovulatory progesterone levels) more than 5 days after taking UPA-EC but before the end of the experimental cycle. The difference between the two experimental arms with respect to the occurrence of ovulation with risk of pregnancy was statistically significant (p=0.042). The risk of OSa was also significantly higher in Arm 2 in the experimental cycle (OR 7.78 [1.38; 43.95], p=0.02) than in Arm 1.

Thus, contrary to current clinical guidelines to wait five days to resume COC, women who use UPA 30 mg for EC after missing COC pills should restart their COC immediately. If they do not, they may be at risk of pregnancy if intercourse occurs during the remainder of their pill pack.

1. A method for providing contraception to a woman who is using a combined oral contraceptive (COC) composition, the method comprising:
(a) administering ulipristal acetate to the woman (i) after the woman has missed at least one daily dose of the COC composition, and (ii) within about 120 hours after the woman has unprotected intercourse in the woman's same menstrual cycle when the woman missed at least one daily dose of the COC composition; and
(b) resuming administration of the COC composition to the woman within about 24 hours after administering ulipristal acetate,
wherein the COC composition comprises an estrogen and a progestogen.

2. The method of claim 1, wherein the unprotected intercourse is within about 120 hours before or after the woman has missed at least one daily dose of the COC composition.

3. The method of claim 1, wherein step (a) comprises administering orally about 30 mg ulipristal acetate to the woman.

4. The method of claim 1, wherein the estrogen comprises ethinyl estradiol, and wherein the progestogen comprises levonorgestrel.

5. The method of claim 1, wherein the woman has missed no more than three consecutive daily doses of the COC composition, wherein step (a) comprises administering ulipristal acetate to the woman within about 24 hours after the woman's last missed daily dose of the COC composition, and wherein step (b) comprises resuming administration of the COC composition within about 18 hours after administering ulipristal acetate.

6. The method of claim 1, wherein the woman has used the COC composition for at least one menstrual cycle before the woman has missed at least one daily dose of the COC composition.

7. The method of claim 1, wherein step (b) comprises resuming administration of the COC composition on a same day after administering ulipristal acetate.

8. The method of claim 1, wherein step (b) comprises resuming administration of the COC composition within about 12 hours after administering ulipristal acetate.

9. The method of claim 1, wherein step (a) comprises administering ulipristal acetate to the woman within about 72 hours after the woman has missed at least one daily dose of the COC composition.

10. The method of claim 1, which decreases the risk of, or inhibits, ovulation in the woman's remaining menstrual cycle.

11. A method for providing contraception to a woman who is using a combined hormonal contraceptive, the method comprising:
(a) administering orally about 30 mg ulipristal acetate to the woman within about 120 hours after the woman has missed at least one daily dose of the combined hormonal contraceptive, and
(b) resuming administration of the combined hormonal contraceptive within about 24 hours after administering ulipristal acetate,
wherein the combined hormonal contraceptive comprises an estrogen and a progestogen.

12. The method of claim 11, wherein the combined hormonal contraceptive is a combined oral contraceptive (COC) composition, a contraceptive patch, or a vaginal ring.

13. The method of claim 12, wherein the woman has used the COC composition for at least one menstrual cycle.

14. The method of claim 13, wherein the woman has missed no more than three consecutive daily doses of the COC composition.

15. The method of claim 12, wherein the estrogen comprises ethinyl estradiol, and wherein the progestogen comprises levonorgestrel.

16. The method of claim 14, wherein step (a) comprises administering ulipristal acetate within about 24 hours after the woman's last missed daily dose of the COC composition.

17. The method of claim 12, wherein step (b) comprises resuming administration of the COC composition within about 18 hours after administering ulipristal acetate.

18. The method of claim 12, wherein step (b) comprises resuming administration of the COC composition to the woman on a same day after administering ulipristal acetate.

19. The method of claim 11, wherein the woman has unprotected intercourse within about 120 hours before administering ulipristal acetate to the woman.

20. The method of claim 11, wherein the woman has used the combined hormonal contraceptive for at least one month.
